(19) United States
(12) Patent Application Publication
(10) Pub. No.: US 2014/0206056 A1
(43) Pub. Date: Jul. 24, 2014

(54) INSTALLATION AND METHOD FOR BIOMASS CONVERSION INTO METHANE

(71) Applicant: Jacob Hendrik Obbo Hazewinkel, Zoetermeer (NL)

(72) Inventor: Jacob Hendrik Obbo Hazewinkel, Zoetermeer (NL)

(73) Assignee: RED PATENT B.V., An Gorssel (NL)

(21) Appl. No.: 13/982,144

(22) PCT Filed: Jan. 31, 2011
(86) PCT No.: PCT/EP2011/051308

§ 371 Date: Oct. 9, 2013

(51) Int. Cl. C12P 5/02

(52) U.S. Cl. CPC C12P 5/023
USPC 435/167; 435/289.1

(57) ABSTRACT

The present invention relates to apparatuses, such as small and medium scale processing plants, for conversion of biomass into methane and other high-grade products such as fertiliser. The present invention further relates to methods and uses of the present apparatuses for conversion of biomass into methane and other high-grade products such as fertiliser. Specifically, the present invention relates to an apparatus for conversion of biomass, the apparatus comprises: a) an acidification reactor (1) of a mixed fluid type reactor for microbial hydrolysis and acidification of biomass b) a methane synthesis reactor (2) of a solid bed reactor type for the anaerobic microbial conversion of acidified biomass into c) a methane synthesis reactor (3) of a mixed fluid type reactor for anaerobic microbial conversion of acidified liquid biomass d) a nitrification reactor (4) for aerobic microbial conversion of NH₄⁺ into NO₃⁻.

The present invention relates to apparatuses, such as small and medium scale processing plants, for conversion of biomass into methane and other high-grade products such as fertiliser. The present invention further relates to methods and uses of the present apparatuses for conversion of biomass into methane and other high-grade products such as fertiliser.

In the Netherlands, the average annual production of biomass is approximately 30,000 tonnes dry matter of which nearly 28,000 tonnes is produced in agriculture. This tonnage represents an energy content of 475 pJ of renewable energy each year equal to 15 billion m³ natural gas. In comparison, in the Netherlands, the use of natural gas was 1400 pJ in 2008.

Part of the annual biomass production is used as marketable products, or raw materials, while the remainder is not, or scarcely, used. A large portion of the annual biomass production which is used eventually results in organic waste streams, and especially wet organic waste streams, such as liquid manure, manure, sewage sludge, domestic vegetable waste, agricultural plant residue or domestic plant residue.

The energy content of the primarily non-used biomass and the organic waste streams of the primarily used biomass is considerable in addition to nutrient content, for example, nitrogen, phosphor, minerals and trace elements.

Conversion of non-used biomass, such as agricultural and forestry waste and organic waste streams of the primarily used biomass into high-grade products, for example natural gas, can significantly contribute to the amount of sustainable energy, or green energy, available for energy consumption and, accordingly, significantly contribute to reduction of green house gasses such as CO₂.

It has been estimated that 30% to 60% of the annual biomass production can be converted into methane or natural gas thereby, for example, providing a renewable potential alternative for 10% to 20% of the natural gas consumption in the Netherlands.

Conversion of organic waste streams, and especially manure, into methane has been used for decades. The most commonly used method basically comprises a large air-sealed holder in which manure is collected and allowed to ferment, i.e. convert or digest, carbon based or organic materials into methane, generally for 30 to 40 days. The resulting (bio)gas generally comprises approximately 40% CO₂, 60% methane and changing amounts of H₂S.

In principle, the (bio)gas produced is not directly suitable for energy consumption because, amongst others, its relatively low methane content. Additionally, the presence of large amounts of CO₂ and H₂S is undesired in an energy source.

In practice, it is been shown that fermentation of manure alone does not suffice to efficiently convert manure into methane. The addition to the manure of additional nutrient sources such as maize is required to aid the fermentation process.

Further, in practice, it has been shown that only large scale biogas production facilities can be economically exploited for biogas production. On site small scale plants using locally produced biomass are not feasible from an investment and production yield point of view.

An additional problem associated with traditional natural gas, or methane, production using biomass is the residue obtained after fermentation. This residue comprises high concentrations of microorganisms, besides ammonia, heavy metals, phosphor and nitrogen, and is not directly suitable to be used, for example as a fertiliser, and, accordingly has to be further processed or disposed thereby, amongst others, increasing the costs of the traditional biogas production process.

A further problem especially associated with organic waste streams produced by animals, such as (liquid) manure, is the annual release of NH₃, or ammonia, in the environment, for example by discarding the manure or directly using it as fertiliser. The discarded animal organic waste streams also significantly contribute to the additional direct release of methane, a green house gas, in the atmosphere.

Although the problems associated with renewable natural gas, or methane, production from biomass have been described above especially in relation to manure conversion into biogas, most of these problems, such as inefficient conversion, low-grade biogas, not feasible on a small scale, are also associated with other organic waste streams such as sewage sludge, domestic vegetable waste, agricultural plant residue or domestic plant residue.

Especially for small scale conversion of biomass, a biomass conversion plant, or installation, preferably meets most, if not all, of the requirements presented below:
Maximal reduction of emissions of acidifying or green house gasses;
All biomass can be processed or converted locally thus not only liquid manure;
Recycling of phosphor, potassium and nitrogen in directly useable high grade products, for example by separating phosphor and potassium and converting NH₃ into NH₄NO₃;
Production of biogas comprising high concentrations of methane;
Production of stabilised organic matter, such as compost, directly useable as fertilizer;
High energy conversion rates;
No, or a significant reduction, of H₂S emissions;
Fully automatable;

Especially for medium scale conversion of biomass, a biomass conversion plant or installation preferably meets most, if not all, of the requirements presented below:
Production of biogas comprising high concentrations of methane;
Recycling of phosphor, potassium and nitrogen in directly useable high grade products, for example by separating phosphor and potassium and converting NH₃ into NH₄NO₃;
Concentrated fertilizer liquids;
Possibility to separate heavy metals;
Production of substantially microbial free stabilized organic matter such as compost;
High energy conversion rates;
No, or a significant reduction, of emissions of acidifying, green house gasses and H₂S emissions;
Cost efficient.

It is an object of the present invention, amongst other objects, to provide apparatuses, or installations or plants, for conversion of biomass into methane and other high-grade products meeting at least part, if not all, of the above requirements for small scale (local) and/or medium scale (regional) production facilities of natural gas and other high-grade products.

The above object, amongst other objects, is met by an apparatus for conversion of biomass as defined appended claim 1.

Specifically, the above object, amongst other objects, is met by an apparatus for conversion of biomass, the apparatus comprises:
a) an acidification reactor (1) of a mixed fluid type reactor for microbial hydrolysis and acidification of biomass comprising at least one intake comprising at least one inlet for receiving biomass, at least one discharge end comprising at least one outlet for discharging acidified biomass, the acidification reactor (1) is operated at a temperature of 45° C. to 70° C. and a pH of 3 to 4.5;
b) a methane synthesis reactor (2) of a solid bed reactor type for the anaerobic microbial conversion of acidified biomass into methane comprising at least one intake for receiving acidified biomass from the acidification reactor (1), at least one discharge end comprising at least three outlets for discharging processed sludge biomass, methane comprising gaseous effluent and acidified liquid biomass, the methane synthesis reactor (2) is operated at a temperature of 20° C. to 60° C., a pH of 6.5 to 8 and a redox potential of −150 mV to −450 mV;
c) a methane synthesis reactor (3) of a mixed fluid type reactor for anaerobic microbial conversion of acidified liquid biomass into methane comprising at least one intake comprising at least one inlet for receiving acidified liquid biomass from the methane synthesis reactor (2) and at least one discharge end comprising at least two outlets for discharging processed liquid biomass and methane comprising gaseous effluent, the methane synthesis reactor (3) is operated at a temperature of 20° C. to 60° C., a pH of 6.5 to 8 and a redox potential of −150 mV to −450 mV;
d) a nitrification reactor (4) for aerobic microbial conversion of NH₄⁺ into NO₃ comprising at least one intake comprising at least one inlet for receiving processed liquid biomass from the methane synthesis reactor (3), at least one discharge end comprising at least one outlet for discharging nitrified processed liquid biomass, the nitrification reactor (4) is operated at a temperature of 15° C. to 37° C., a pH of 6.5 to 7.5 and a redox potential of more than 50 mV.

The present inventors have surprisingly found that the above combination and order of separate reactors operated under the conditions specified, provides:
biogas comprising high concentrations of methane (approximately 60%). Because of the composition of the biogas produced (relatively free from interfering contaminants), the biogas can be easily further processed into biogas comprising 90 to 99.8% methane, for example using standard techniques such as a potassium carbonate wash or cryogenic distillation. Further, the present biogas produced comprises less than 2 ppm H₂S;
the high-grade fertilisers are produced, i.e., nitrified processed liquid biomass and processed sludge biomass, having a pH of around 7 and not comprising gaseous organic matter allowing them to be directly used as fertilizer.

The present reactors (1) to (4) are based on microbial conversion, or processing, of biomass. The microorganisms, such as fungi and bacteria, used in the reactors can be provided by, or present in, the biomass itself, or can be inoculated in the reactors at, for example, start-up of the apparatus. Suitable inoculation cultures can be found in waste and surface water purification installations.

According to the present invention, selection of species of microorganisms is not particularly important. The reaction conditions defined allow the creation of specific environments favouring the growth and/or phenotype of acid producing microorganisms, such as fungi, in the acidification reactor (1), production of methane, for example by bacteria, in the methane synthesis reactors (2) and (3) and the nitrification in the nitrification reactor (4).

The present acidification reactor (1) of a mixed fluid type reactor substantially provides acidification by acid secretion of microorganisms. However, for example, when the biomass supplied comprises a high nitrogen content, the indicated pH range can be optionally maintained by adding additional sugar or acid to the biomass or into the reactor (1).

The present inventors have surprisingly found that by microbial acidification of the biomass under the conditions specified:
a phase separation occurs between an acidified liquid biomass comprising dissolved minerals and dissolved organic compounds such as acetic acid and hydrocarbon breakdown products, and acidified sludge biomass comprising, for example, fibre-like materials and minerals such as sand and clay;
a surface layer is formed substantially comprised of plastics and/or lignocelluloses.

If present, contaminants such as plastics can be readily removed from the process stream by separation, or isolation, of the surface layer.

The above phase separation allows separating sludge and liquid process flows using traditional techniques such as sedimentation, filtration, tilted plate separators, or crossflow microfiltration. Additionally, separated sludge and liquid flows prevent clogging of the apparatus and allow efficient heat-exchange providing a reduction of external heat required by 60% to 70%.

After acidification of the biomass, the acidified biomass is transported to and discharged in a methane synthesis reactor (2) allowing separation of the acidified biomass in a sludge and liquid stream. The acidified sludge biomass is subjected to an anaerobic environment allowing microbial methane production and the acidified liquid biomass is discharged into a methane synthesis reactor (3) where it is separately subjected to a similar anaerobic environment allowing microbial methane production.

Thereafter, the processed liquid biomass is transported to and discharged in a nitrification reactor (4). Under the conditions specified, the nitrification reactor (4) microbially converts NH₄⁺ (NH₃) into non-gaseous NO₃⁻ thereby lowering the pH of the processed liquid biomass to a pH of 6.5 to 7.5 resulting in a directly useable, for example as a liquid fertilizer solution, neutral mixture of ammonium nitrate and urea.

The present inventors have surprisingly found that the apparatus as described above allows conversion of biomass in 1 to 2 days, in comparison, the traditional plants require 30 to 40 days, with an efficiency of conversion of 80 to 85% per day or more.

Without being limiting to the invention because of an underlying mechanism, at least a substantial part of the efficiency of the present apparatus with respect to methane production appears to be attributable to high concentrations of acetic acid in the acidified liquid biomass.

According to a preferred embodiment of the present invention, the present acidification reactor (1) further comprises an outlet for discharging H₂S comprising gaseous effluent and the apparatus further comprises:
e) an effluent gas conversion reactor (5) for aerobic microbial conversion of H₂S into SO₄²⁻ comprising at least one intake comprising at least inlet for receiving H₂S comprising gaseous effluent from the acidification reactor (1) and at least one discharge end comprising at least one outlet for discharging CO₂, H₂O, SO₄²⁻, the effluent gas conversion reactor (5) is operated at a temperature of 15° C. to 35° C., a pH of 3.0 to 4.5 and a redox potential of more than 50 mV;

The present inventors have surprisingly found that the gaseous effluent of the acidification reactor (1) substantially comprises a substantial amount of, if not all, sulphur in the form of H₂S present in the biomass supplied. Accordingly, substantially all sulphur, or at least a significant portion thereof, can be conveniently removed in a early stage of the conversion process by discharging the gaseous effluent from the acidification reactor (1).

By transporting and discharging the gaseous effluent in the present effluent gas conversion reactor (5) and subjecting it to the condition specified, microbial conversion of gaseous H₂S into SO₄⁻ salts is obtained.

Acidic liquid comprising SO₄⁻ discharged from the effluent gas conversion reactor (5) can be conveniently used in the apparatus for pH regulation.

According to another preferred embodiment of the present invention, the apparatus for conversion of biomass comprises:
f) a composting reactor (6) for microbial decomposition of biomass comprising at least one intake comprising at least one inlet for receiving processed sludge biomass from the methane synthesis reactor (2) and at least one discharge end comprising at least one outlet for discharging composted biomass, the composting reactor (6) is operated at a temperature of 45° C. to 75° C., a pH and an oxygen concentration of 2 to 20%.

The present composting reactor (6) receives processed sludge biomass from the methane synthesis reactor (2) and subjects the sludge to the indicated conditions for a period of time sufficient for drying and further digestion, such as for 10 to 30 days.

The controlled oxygen pressure and relatively high temperature ensures efficient composition. In addition, at least partially performing the process at temperatures above 70° C. allows for decontaminating the compost of most potential pathogenic microorganisms.

Since the input stream of the composting reactor (6) is low in sulphur, sulphur is removed in the acidification reactor (1), but high in minerals and trace elements, the resulting composted biomass is a high-grade directly usable fertiliser.

If present in the biomass, heavy metals can be readily removed by subjecting the acidified or processed sludge biomass to a sedimentation step and removing the sediment comprising heavy metals from the process stream(s).

According to yet another preferred embodiment, the present invention relates to an apparatus for conversion of biomass wherein the composting reactor (6) comprises a further outlet for discharging acetate comprising leachate and the acidification reactor (1) comprises a further inlet for receiving the acetate comprising leachate.

Acetate or acetic acid comprising leachate is produced in the composting reactor (6) as a hydrolyzation product of cellulose. Because of the relatively mild acidic nature of acetic acid, in addition to its buffering capacities, the leachate produced by the composting reactor (6) can be transported to, and discharged in, the acidification reactor (1) thereby assisting in maintaining the pH in the required range.

Gaseous effluent from the composting reactor (6) comprising NH₃ can be conveniently processed in the nitrification reactor (4).

According to still another preferred embodiment, the present invention relates to an apparatus wherein the methane synthesis reactor (2) comprises a phase separation device for separating the acidified biomass into an acidified liquid biomass and an acidified sludge biomass comprising at least one inlet for receiving acidified biomass and at least two outlets for discharging acidified liquid biomass to the methane synthesis reactor (3) and acidified sludge biomass and a methane synthesis device comprising at least one inlet for receiving the acidified sludge biomass and at least one outlet for discharging processed sludge biomass, the methane synthesis device is operated at a temperature of 20° C. to 60° C., a pH of 6.5 to 8 and a redox potential of −150 mV to −450 mV

As indicated, the acidification of the biomass yields, amongst others, phase separation of the acidified biomass. Accordingly, in a preferred embodiment, phase separation of acidified liquid and sludge biomass is performed before methane synthesis by microbial conversion.

According to a further preferred embodiment, the present apparatus comprises between the methane synthesis reactor (3) and the nitrification reactor (4) a vacuum device for concentrating the processed liquid biomass, the vacuum device comprises an inlet for receiving processed liquid biomass from the methane synthesis reactor (3) and an outlet for discharging concentrated processed liquid biomass to the nitrification reactor (4), an outlet for discharging CO₂ and methane and an outlet for discharging CaCO₃ and NH₄MgPO₄, the vacuum device is operated to subject the processed liquid biomass to a vacuum of 20 to 200 mbar until a pH of at least 8, preferably at least 8.5.

By subjecting processed liquid biomass to a vacuum not only the dissolved methane is extracted, thereby increasing the yield of the present apparatus, but also dissolved CO₂. By extracting CO₂, the pH of the processed liquid biomass increases to the indicated range and, as a result, phosphor and magnesium in the form of NH₄MgPO₄ and calcium in the form of CaCO₃ precipitates and, accordingly, can be conveniently removed from the process stream as a high-grade product.

NH₄MgPO₄ and CaCO₃ removed can be brought to a substantially neutral pH, for example by using the SO₄⁻ from the effluent gas conversion reactor (5), yielding a directly marketable product.

As indicated, the present apparatus is substantially kept in homeostasis, after start-up, for the indicated process conditions by microorganisms. However, for example depending on the type of biomass supplied to the apparatus, process conditions can deviate from the indicated conditions, for example, the pH in the acidification reactor can vary depending on the nitrogen content of the biomass supplied.

Accordingly, according to a preferred embodiment, the present apparatus comprises a controlling device to monitor the indicated pHs, temperatures and/or the redox potentials, and preferably, further comprises reactors, where appropriate, provided with heating devices for maintaining the temperature in the defined range, with pH regulating devices for maintaining the pHs in the defined range, and redox potential regulating devices for maintaining the redox potential in the defined range.

Temperature regulating devices can be heaters providing heat generated or derived from the apparatus itself, or heat from an external source, coolers providing cooling generated or derived from the apparatus itself, or cooling from an external source.

pH regulating devices can be holders comprising sugar, buffer, acid or basic liquid fitted with supply means for introducing the sugar, buffer, acid or basic liquid in the appropriate reactor, and/or a transport system controlling the flow of basic or acidic fluids in the apparatus itself, for example the leachate produced by the composting reactor (6).

Redox potential regulating devices can be holders comprising liquids with a defined redox potential fitted with supply means for introducing the liquids in the appropriate reactor.

According to still another preferred embodiment, the present one or more communicating inlets and outlets of the reactors comprise devices for isolation of microorganisms and for reintroducing the isolated microorganisms in the reactors from which they were derived from.

In other words, the microorganisms in a reactor inherently discharged with the process flows are continuously reintroduced into the reactor thereby providing a stable culture of microorganisms in the reactor, and, accordingly, a stable control of methane synthesis and other microbial processes.

The present apparatus is particularly suitable to process biomass, especially to convert biomass into methane and/or fertilizer, selected from the group consisting of liquid manure, manure, sewage sludge, domestic vegetable waste, agricultural plant residue, domestic plant residue, and combinations thereof.

As indicated above, methane and other high-grade products can be conveniently collected at the outlets of reactors comprised in the present apparatus. Accordingly, according to a preferred embodiment, the present invention relates to an apparatus wherein the methane is collected at the outlets of the methane synthesis reactor (2) and the methane synthesis reactor (3) and the fertilizer at the outlets of the nitrification reactor (4) and/or the composting reactor (6).

The apparatus as described above provides an efficient conversion of (waste) biomass into valuable products. Therefore, according to another aspect, the present invention relates to a method for conversion of biomass comprising:
a) supplying the acidification reactor (1) of the present apparatus with biomass;
b) operating:
   1) the acidification reactor (1) at a temperature of 45° C. to 70° C. and a pH of 3 to 4.5;
   2) the methane synthesis reactor (2) at a temperature of 20° C. to 60° C., a pH of 6.5 to 8 and a redox potential of −150 mV to −450 mV under anaerobic conditions;
   3) the methane synthesis reactor (3) at a temperature of 20° C. to 60° C., a pH of 6.5 to 8 and a redox potential of −150 mV to −450 mV under anaerobic conditions;
   4) optionally, the effluent gas conversion reactor (5) at a temperature of 15° C. to 35° C., a pH of 3.0 to 4.5 and a redox potential of more than 50 mV under aerobic conditions;
   5) the nitrification reactor (4) at a temperature of 15° C. to 37° C., a pH of 6.5 to 7.5 and a redox potential of more than 50 mV under aerobic conditions;
      wherein acidified biomass is transported from the acidification reactor (1) to the methane synthesis reactor (2), acidified liquid biomass is transported from the methane synthesis reactor (2) to the methane synthesis reactor (3), H₂S comprising gaseous effluent is transported from the acidification reactor (1) to the effluent gas conversion reactor (5), processed liquid biomass is transported from the methane synthesis reactor (3) to the nitrification reactor (4).

According to a preferred embodiment of the present method, the processed sludge biomass from the methane synthesis reactor (2) is transported to a composting reactor (6) operated at a temperature of 45° C. to 75° C., a pH and an atmospheric air concentration of 2 to 20%.

According to another preferred embodiment of the present method, acetate comprising leachate is transported from the composting reactor (6) to the acidification reactor (1).

According to still another preferred embodiment of the present method, the biomass is selected from the group consisting of liquid manure, manure, sewage sludge, domestic vegetable waste, agricultural plant residue, domestic plant residue, and combinations thereof.

According to a further preferred embodiment of the present method, the conversion of biomass comprises conversion of biomass into methane and/or fertilizer.

The apparatuses and methods as described above provide an efficient conversion of (waste) biomass into valuable products. Therefore, according to another aspect, the present invention relates to use of the present apparatuses for conversion of biomass, preferably the biomass is selected from the group consisting of liquid manure, manure, sewage sludge, domestic vegetable waste, agricultural plant residue, domestic plant residue, and combinations thereof.

According to a preferred embodiment, the present use results in the conversion of biomass into methane and/or fertilizer.

1. An apparatus for converting biomass, the apparatus comprising:
a) an acidification reactor of a mixed fluid type reactor for microbial hydrolysis and acidification of biomass, comprising an intake comprising an inlet for receiving biomass, and a discharge end comprising an outlet for discharging acidified biomass,
wherein the acidification reactor is operated at a temperature of from 45° C. to 70° C. and a pH of from 3 to 4.5;
b) a methane synthesis reactor of a solid bed reactor type for anaerobic microbial conversion of the acidified biomass into methane, comprising an intake for receiving the acidified biomass from the acidification reactor and a discharge end comprising three outlets for discharging processed sludge biomass, methane comprising gaseous effluent and acidified liquid biomass,
wherein the methane synthesis reactor is operated at a temperature of from 20° C. to 60° C., a pH of from 6.5 to 8, and a redox potential of from −150 mV to 450 mV;
c) an additional methane synthesis reactor of a mixed fluid type reactor for the anaerobic microbial conversion of the acidified liquid biomass into methane comprising an intake comprising an inlet for receiving the acidified liquid biomass from the methane synthesis reactor and a discharge end comprising two outlets for discharging processed liquid biomass and the methane comprising gaseous effluent,
wherein the additional methane synthesis reactor is operated at a temperature of from 20° C. to 60° C., a pH of from 6.5 to 8, and a redox potential of from −150 mV to −450 mV; and
d) a nitrification reactor for aerobic microbial conversion of NH4⁺ into NO₃⁻, comprising an intake comprising an inlet for receiving the processed liquid biomass from the additional methane synthesis reactor, and a discharge end comprising an outlet for discharging nitrified processed liquid biomass,
wherein the nitrification reactor is operated at a temperature of from 5° C. to 37° C., a pH of from 6.5 to 7.5 and a redox potential of more than 50 mV.

2. The apparatus according to claim 1, the apparatus further comprises:
e) an effluent gas conversion reactor for aerobic microbial conversion of H₂S into SO₄²⁻, comprising an intake comprising an inlet for receiving H₂S comprising gaseous effluent from the acidification reactor and a discharge end comprising an outlet for discharging CO₂, H₂O, and SO₄²⁻,
wherein the effluent gas conversion reactor is operated at a temperature of from 15° C. to 35° C., a pH of from 3.0 to 4.5 and a redox potential of more than 50 mV,
wherein the acidification reactor further comprises an additional outlet for discharging the H₂S comprising gaseous effluent.

3. The apparatus according to claim 1, the apparatus further comprising:
f) a composting reactor for microbial decomposition of processed sludge biomass, comprising an intake comprising an inlet for receiving the processed sludge biomass from the methane synthesis reactor and a discharge end comprising an outlet for discharging composted biomass,
wherein the composting reactor is operated at a temperature of from 45° C. to 75° C., a pH and an oxygen concentration of from 2 to 20%. 25.

4. The apparatus according to claim 3,
wherein the composting reactor comprises an additional outlet for discharging acetate comprising leachate and
the acidification reactor comprises an additional inlet for receiving the acetate comprising leachate from the composting reactor.

5. The apparatus according to claim 1,
wherein the methane synthesis reactor comprises a phase separation device for separating the acidified biomass into the acidified liquid biomass and an acidified sludge biomass, the phase separation device comprises an inlet for receiving the acidified biomass and two outlets for discharging acidified liquid biomass to the methane synthesis reactor and the acidified sludge biomass, and
a methane synthesis device comprising an inlet for receiving the acidified sludge biomass from the phase separation device and an outlet for discharging the processed sludge biomass, the methane synthesis device is operated at a temperature of from 20° C. to 60° C., a pH of from 6.5 to 8 and a redox potential of from −150 mV to −450 mV.

6. The apparatus according to claim 1, further comprising between the additional methane synthesis reactor and the nitrification reactor:
a vacuum device for concentrating the processed liquid biomass,
wherein the vacuum device comprises an inlet for receiving the processed liquid biomass from the additional methane synthesis reactor and an outlet for discharging concentrated processed liquid biomass to the nitrification reactor, an outlet for discharging CO₂ and methane and an outlet for discharging CaCO₃ and NH₄MgPO₄, and
wherein the vacuum device is operated to subject the processed liquid biomass to a vacuum of from 20 to 200 mbar until a pH of at least 8.

7. The apparatus according to claim 1, further comprising:
a controlling device to monitor pH, a temperature, a redox potential, or a combination thereof.

8. The apparatus according to claim 7,
wherein the acidification reactor, the methane synthesis reactor, the additional methane synthesis reactor, the nitrification reactor, and the effluent gas conversion reactor comprise heating devices for maintaining a temperature in a defined range, having a pH regulating devices device for maintaining a pH in a defined range, and
the methane synthesis reactor, the additional methane synthesis reactor, the nitrification reactor, and the effluent gas conversion reactor comprise a redox potential regulating device for maintaining a redox potential in a defined range.

9. The apparatus according to claim 1, wherein communicating inlets and outlets of the acidification reactor, the methane synthesis reactor, the additional methane synthesis reactor, and the nitrification reactor comprise devices for isolating a microorganism and reintroducing an isolated microorganism in the acidification reactor, the methane synthesis reactor, the additional methane synthesis reactor, and the nitrification reactor from which it was derived from.

10. The apparatus according to claim 1, wherein the biomass is at least one selected from the group consisting of liquid manure, manure, sewage sludge, a domestic vegetable waste, an agricultural plant residue, and a domestic plant residue.

11. The apparatus according to claim 1, wherein the converting comprises converting into methane, fertilizer, or a combination thereof.

12. The apparatus according to claim 11, wherein the methane is collected at the three outlets of the methane synthesis reactor and the additional methane synthesis reactor and
the fertilizer is collected at the outlet of the nitrification reactor, the composting reactor, or a combination thereof.

13. A method for converting biomass, the method comprising:
a) supplying the acidification reactor of the apparatus according to claim 1 with biomass;
b) operating:
1) the acidification reactor at a temperature of from 45° C. to 70° C. and a pH of from 3 to 4.5;
2) the methane synthesis reactor at a temperature of from 20° C. to 60° C., a pH of from 6.5 to 8 and a redox potential of from −150 mV to −450 mV under an anaerobic conditions condition;
3) the additional methane synthesis reactor at a temperature of from 20° C. to 60° C., a pH of from 6.5 to 8 and a redox potential of from −150 mV to −450 mV under an anaerobic conditions condition;
4) optionally, an effluent gas conversion reactor at a temperature of from 15° C. to 35° C., a pH of from 3.0 to 4.5 and a redox potential of more than 50 mV under an aerobic condition;
5) the nitrification reactor at a temperature of from 15° C. to 37° C., a pH of from 6.5 to 7.5 and a redox potential of more than 50 mV under an aerobic condition;
wherein the acidified biomass is transported from the acidification reactor to the methane synthesis reactor,
the acidified liquid biomass is transported from the methane synthesis reactor to the additional methane synthesis reactor,
H₂S comprising gaseous effluent is transported from the acidification reactor to the effluent gas conversion reactor,
processed liquid biomass is transported from the additional methane synthesis reactor to the nitrification reactor.

14. The method according to claim 13, wherein the processed sludge biomass from the methane synthesis reactor is transported to a composting reactor operated at a temperature of from 45° C. to 75° C., a pH and an oxygen concentration of from 2 to 20%.

15. The method according to claim 14, wherein acetate comprising leachate is transported from the composting reactor to the acidification reactor.

16. The method according to claim 13, wherein the biomass is at least one selected from the group consisting of liquid manure, manure, sewage sludge, a domestic vegetable waste, an agricultural plant residue, and a domestic plant residue.

17. The method according to claim 13, wherein the converting comprises converting into methane, fertilizer, or a combination thereof.

18. A method for converting biomass, comprising:
Converting the biomass with the apparatus according to claim 1.

19. The method according to claim 18, wherein the biomass is at least one selected from the group consisting of liquid manure, manure, sewage sludge, a domestic vegetable waste, an agricultural plant residue, and a domestic plant residue.

20. The method according to claim 18, wherein the converting comprises converting into methane, fertilizer, or a combination thereof.
